(19) United States
(12) Patent Application Publication
(10) Pub. No.: US 2020/0047384 A1
(43) Pub. Date: Feb. 13, 2020

(54) DEVICE FOR THE CONTINUOUS PRODUCTION OF SOLID DOSAGE FORMS, AND USE OF SAID DEVICE FOR MEDICINAL DRUGS AND/OR USE THEREOF FOR FOOD SUPPLEMENTS

(71) Applicant: Université de Liège, Liège (BE)

(72) Inventors: Justine Thiry, Fooz (BE); Fabrice Krier, Liege (BE); Brigitte Evrard, Liege (BE); Alain Nuozzi, Awans (BE); Carl Emmerechts, Huy (BE); Eric Beeckman, Tongeren (BE)

(21) Appl. No.: 16/340,625

(22) PCT Filed: Sep. 28, 2017
(86) PCT No.: PCT/EP2017/074679

§ 371 Date: Apr. 9, 2019

(30) Foreign Application Priority Data
Oct. 10, 2016 (BE) 201605752

(51) Int. Cl. B29C 48/00; A23P 30/20; A23L 33/105; A23P 10/00; B29C 48/88; B29C 48/06; A61K 31/496; A61K 9/00

(52) U.S. Cl. CPC B29C 48/0022; A23P 30/20; A23L 33/105; A23P 10/00; B29C 48/911; B29K 2105/0035; A61K 31/496; A61K 9/0053; A23V 2002/00; B29C 2793/009; B29C 48/06

(57) ABSTRACT

A device for the continuous production of a solid form, including: an extruder for mixing and hot-melting at least one component with at least one polymer for an amorphous solution; and a cutting device mounted downstream of the extruder, for cutting the semi-solid bead that results from the melting of the mixture of the component with the polymer for an amorphous solid solution, the cutting device being combined with a forming system for forming the solid form supplied by the cutting device.

Device for the continuous production of solid dosage forms, and use of said device for medicinal drugs and/or use thereof for food supplements

The present invention relates to a device for the continuous production of solid forms for pharmaceutical use or for food supplements. More particularly this device is suitable for the continuous production of oral solid dosage forms comprising an active substance of class BCS II of the biopharmaceutics classification system (BCS), which is characterized by low water solubility.

Just like the active principles in class BCS II, food supplements, and more particularly plant extracts such as curcumin and berberine, may also present problems of water solubility.

To overcome low bioavailability of the active substance or of the food supplement, both of which are also called components in the present invention, a person skilled in the art is familiar with dispersion of the component in an amorphous solid polymer matrix to form a single-phase system, called an amorphous solid solution.

“Amorphous solid solution” means a system in the solid state in which the component is dispersed at the molecular level in a matrix in such a way that the system is chemically and physically uniform or homogeneous throughout.

These amorphous solid dispersions in solid form are generally prepared by batch methods of hot extrusion.

In CA 2 209 943, Zetler describes the preparation of oral solid forms by hot melting, produced by mixing and melting at least one pharmacologically acceptable polymer and at least one active substance with or without conventional excipients. The forming of the oral solid form takes place in two additional steps, further to extrusion. An optical system combined with a chopping system allows the rod or extrudate to be measured and fractionated into cylindrical shapes of the desired length. These cylindrical shapes are transported on a conveyor belt and are then rounded off in a subsequent step in a semisolid or plastic state by means of jaws of concave shape. A succession of devices with various functions is therefore required for implementing the forming process, notably the use of jaws for rounding the extrudates. Applying these jaws on tablets still in the semisolid state has drawbacks for the final shape of the solid dosage form. In fact, rounding of the solid dosage forms cannot overcome surface irregularities resulting from a partial adhesion of the molten mass to the chopping system and the resultant loss of material.

The present invention relates to a device for continuous production of solid dosage forms that overcomes these problems. The device according to the invention allows the production of a uniform solid dispersion required for preparing amorphous systems comprising a component, which may be for example an active principle or a food supplement with low solubility in a solvent, preferably an aqueous solvent.

The device makes it possible to improve the dissolution of the component from the oral solid form obtained and its bioavailability. This results in a lower concentration of the components in the oral solid form and a concentration that is more suitable for the pharmaceutical application or application as food supplement.

The present invention also makes it possible to overcome imperfections of shape, composition and stability of the solid dosage forms during conventional extrusion processes.

The device according to the invention allows continuous production of solid dosage forms that are perfectly uniform and stable, at all production rates, but preferably at a fast rate of at least 1 oral form per second.

The device also allows real-time monitoring of the critical production parameters by possible implementation of process analytical technology (PAT) based on vibrational spectroscopy. This on-line analysis of the product is rapid, nondestructive, and solvent-free and allows continuous feedback for the production parameters.

The continuous device according to the invention comprises a combination of an extruder or a hot kneader and a continuous direct forming system, preferably a hot forming system.

The conventional extruder or kneader allows hot melting of the mixture of at least one component with a polymer for amorphous solution and, in addition, continuous delivery of a molten mass that can be supplied in single elements of constant masses and shapes that are suitable for example for oral administration.

An extruder according to the invention may be a single-screw extruder, an intermeshing-screw extruder, an extruder with several corotating or counterrotating screws, optionally equipped with kneading disks. A person skilled in the art will appreciate that the energy to be applied to the process and device according to the invention depends on the type of extruder or type of screw configuration that is used. Part of the energy required for melting, mixing and dissolving the components in the extruder may be supplied by the heating elements. However, friction and shearing of the material in the extruder may also supply a substantial amount of energy to the mixture and aid the formation of an amorphous homogeneous molten mass of the component or components with the polymer or polymers for amorphous solution.

For a twin-screw extruder, the rotary speed of the endless screw is preferably from 50 to 300 revolutions per minute. The temperature in the extrusion zone and the temperature of the die are preferably in the range from 50 to 250° C.

In a preferred embodiment of the invention, the device uses a hot-melting extruder in which an active substance or food supplement is introduced and mixed with at least one thermoplastic polymer for amorphous solution, preferably a water-soluble thermoplastic polymer for hot melting of the mixture obtained. Melting involves heating the mixture to a temperature above room temperature and near the glass transition temperature (Tg) of the polymer for amorphous solution, in particular a water-soluble thermoplastic polymer. The extruder may be used for a step of mixing the component with the polymer for amorphous solution prior to melting or else a step of mixing simultaneously with melting. In general, the molten mass obtained is homogenized hot in order to disperse the component or components efficiently in the amorphous matrix. The molten mass is semisolid or pasty.

The polymer chosen for amorphous solution is preferably a water-soluble polymer and must preferably meet certain criteria regarding its glass transition temperature (T_{g}):
1. T_{g} above room temperature (Tᵣₒₒₘ) so that it is in solid form
2. T_{g} at least 50° C. below the degradation temperature of the polymer (i.e. its temperature of change of chemical nature)
3. T_{g} below 200° C. so that the extrusion temperature (i.e. temperature of the molten mass) is below 250° C.

The polymers for amorphous solution, in particular the water-soluble thermoplastic polymers having a T_{g} as defined above, allow preparation of solid dispersions that are mechanically stable at room temperature, so that the amorphous solid dispersions or solutions can be used as dosage forms without additional treatment.

The polymer for amorphous solution is for example a homopolymer or copolymer of N-vinyllactam, in particular a homopolymer or copolymer of N-vinylpyrrolidone, such as polyvinylpyrrolidone (PVP), a copolymer of N-vinylpyrrolidone and vinyl acetate or a vinyl propionate, or a copolymer of polyethylene glycol grafted with chains of polyvinylcaprolactam and polyvinyl acetate or any combination thereof.

The polymer for amorphous solution may also be a cellulose ester or cellulose ether, in particular methylcellulose and ethylcellulose, hydroxyalkylcelluloses, notably hydroxypropylcellulose, hydroxyalkylalkylcellulose, in particular hydroxypropylmethylcellulose, a cellulose phthalate or a succinate, in particular a cellulose acetate phthalate and a hydroxypropylmethylcellulose phthalate, a succinate, or hydroxypropylmethylcellulose acetate succinate or any combination thereof;

The polymer for amorphous solution may also be a polyalkylene oxide of high molecular weight such as an ethylene oxide or polypropylene oxide or a copolymer of ethylene oxide or of propylene oxide, or any combination thereof.

Finally, the water-soluble polymer for amorphous solution may also be a polyacrylate or polymethacrylate such as a copolymer of methacrylic acid/ethyl acrylate, methacrylic acid/methyl methacrylate, butyl methacrylate, a methacrylate of copolymers/2-dimethylaminoethyl, a poly(hydroxyalkyl acrylate), a poly(hydroxyalkyl methacrylate), a polyacrylamide, a vinyl acetate polymer such as the copolymers of vinyl acetate and crotonic acid, partially hydrolyzed vinyl acetate (also called partially saponified “polyvinyl alcohol”), polyvinyl alcohol, or any combination thereof.

Depending on the intended application, the polymer for amorphous solution, in particular the water-soluble polymer, will be of pharmaceutical grade for oral dosage forms or of a grade that meets the purity criteria requested by the food industry for food supplements. A person skilled in the art will refer to the relevant legal provisions for complying with these criteria.

The thermoplastic polymer for amorphous solution is preferably a poly(ethylene glycol) grafted with a copolymer of polyvinyl caprolactam and polyvinyl acetate for the oral forms for pharmaceutical applications and a poly(butylnnethacrylate-co-(2-dinnethylanninoethyl)nnethacrylate-co-methyl methacrylate polymer for the oral forms for food applications.

The amorphous solution or molten mass or rod may be obtained under the action of an extruder screw propelling the mixture of the components with the polymer matrix in the semisolid molten state through an extrusion die.

According to a preferred embodiment of the invention, the device comprises a chopping device arranged downstream of an extruder for delivering the appropriate solid dosage forms starting from the extruded rod. The chopping device is preferably combined with a rod cooling system, which is itself arranged on the “forming” system.

The system for surface cooling of the rod makes it possible to limit its risk of adhesion, even partial, to the chopping device first, and then to the forming system. The decrease in this adhesion to the chopping device may be further reinforced by a particular stepped design of the discharge surface of the extruder die.

Combining this system for cooling the molten mass or rod as it leaves the extruder die with the system for forming the solid form therefore helps to prevent the irregularities of the solid form that result from this adhesion and a possible excess or loss of material in connection with adhesion.

The chopping device comprises an actuating system activated by a motor, preferably linear. The actuating system is specially designed so that the rate of movement of the rod chopping means is fast enough for chopping the extrudate instantaneously and detaching it from the extruder without adherence to the chopping means. This device is far quicker than the rotating knife systems that are familiar to a person skilled in the art.

Preferably the speed of the chopping means and the frequency of actuation of the motor are set respectively at between 0.1 and 1 m per second, preferably 0.3 m per second, for a frequency of at least one solid form per second. The actuating system of the present invention has been developed to allow rapid reciprocating motion of the chopping means, which severs the rod and then detaches it during its return movement. The return movement of the chopping means has the particular feature of going around the die orifice so as not to touch the rod after it has been chopped.

In a particular embodiment, the present invention relates to the use of the device for preparing oral solid forms comprising a solid dispersion of at least one active substance of class BCS II in a pharmaceutical-grade water-soluble thermoplastic polymer.

For this use, the pharmaceutical-grade water-soluble polymer must meet certain criteria regarding its glass transition temperature (T_{g}):
1) T_{g} above room temperature (Tᵣₒₒₘ) so that it is in solid form;
2) T_{g} at least 50° C. below the degradation temperature of the polymer (T_{degradation}) in order to prevent degradation of the polymer during the extrusion process;
3) T_{g} below 200° C. so that the extrusion temperature, i.e. the temperature of the molten mass, is below 250° C.

In a preferred embodiment of the present invention, the use of the device relates to the preparation of oral solid forms comprising itraconazole as active substance of class BCSII.

Itraconazole (also known by the name cis-4{4-(4-(4((2-(2,4-dichlorophenyl)-2-(1H-1,2,4-triazol-1-ylmethyl)-1,3-dioxolan-4yl)nnethoxy)phenyl)-1-piperazinyl)phenyl)-2,4-dihydro-2(1-nnethylpropyl)-3H-1,2,4-triazol-3-one) is a triazole antifungal agent with a piperazine group. Itraconazole has very low water solubility, below 1 microgram per milliliter. It is an example of class BCSII with low solubility and high permeability via the gastrointestinal tract.

In another particular embodiment of the invention, the use of the device relates to the preparation of solid oral forms comprising a compound that belongs to the class of food supplements, for example such as a plant extract or any combination of these supplements. The food supplement is for example curcumin or diferuloyl-methane extracted from the curcuma plant, also called turmeric.

The device and its use according to the invention will now be illustrated with figures and nonlimiting examples in a particular embodiment of the invention.

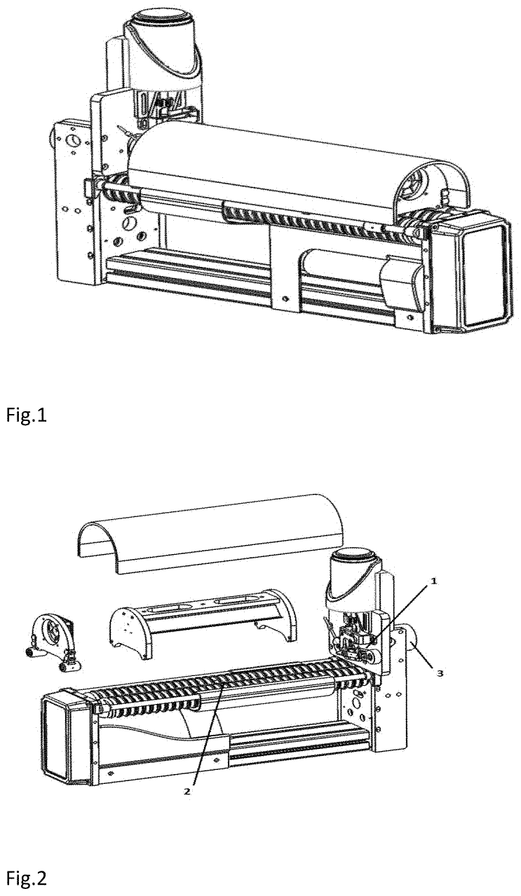

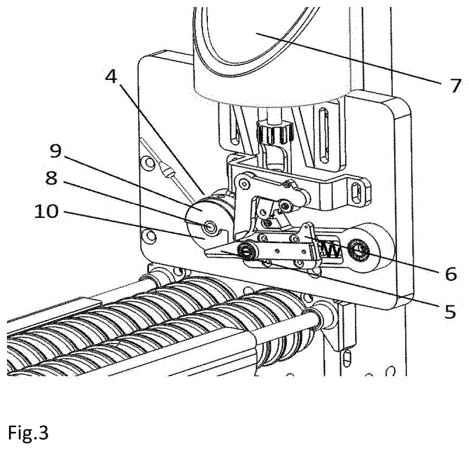

FIGS. 1 and 2 show a general view of the device according to the invention.

FIG. 1 shows a general view of the device according to the invention with an extruder downstream (not shown).

FIG. 2 illustrates a device according to the invention with an exploded view of the cover, revealing a chopping device (1) associated with an orifice (8) of the die of an extruder (3) and a forming system (2) consisting of two corotating Archimedean screws. A detachable heating system is mounted on the two Archimedean screws of the forming system.

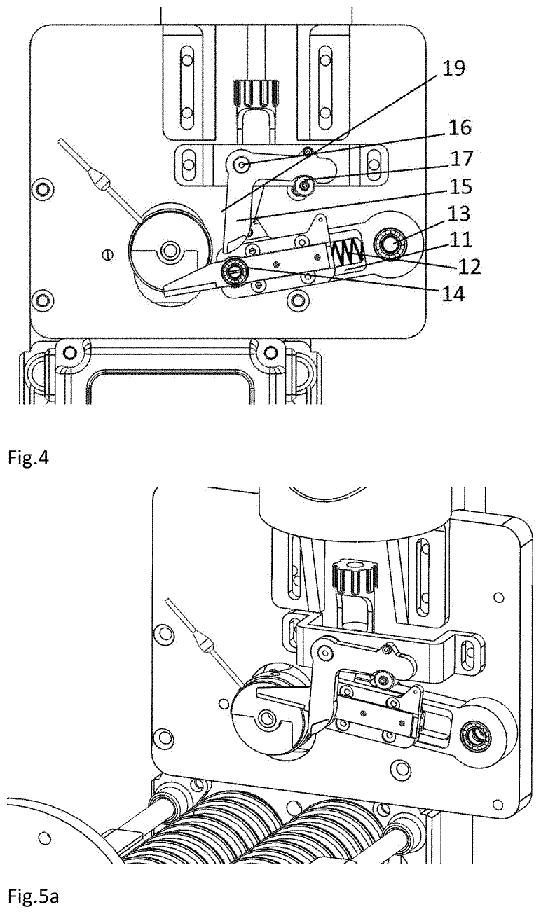

FIG. 3 shows a side view of the chopping device

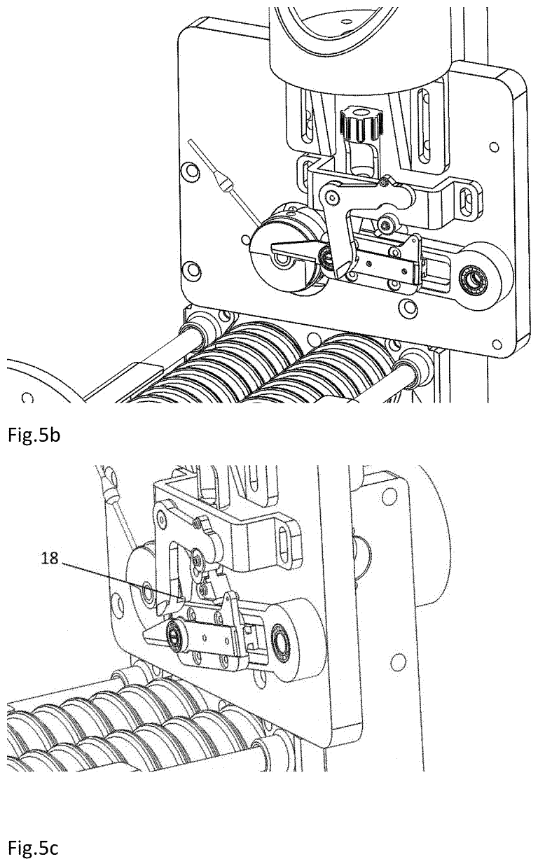

FIG. 4 shows a general front view of the chopping device

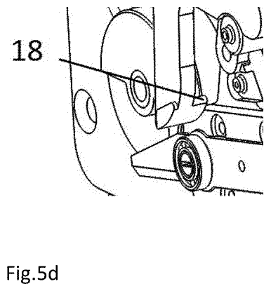

FIG. 5 illustrates the actuating mechanism. FIG. 5a shows the initial position of the chopping means or knife. FIG. 5b shows the position of the knife before chopping the rod.

FIG. 5c shows the knife after chopping. FIG. 5d is a general view showing the shoulder of the pawl responsible for the chopping means or knife going round the rod. FIG. 5e illustrates the movement of the knife in its sliding arm during the downward and upward movement of the knife going round the rod.

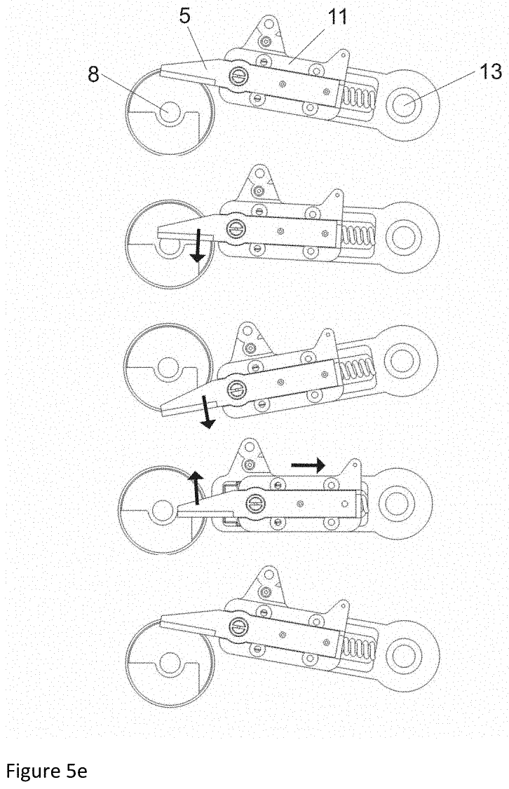

FIG. 6 illustrates the system for cooling the surface of the rod by means of an annular air jet.

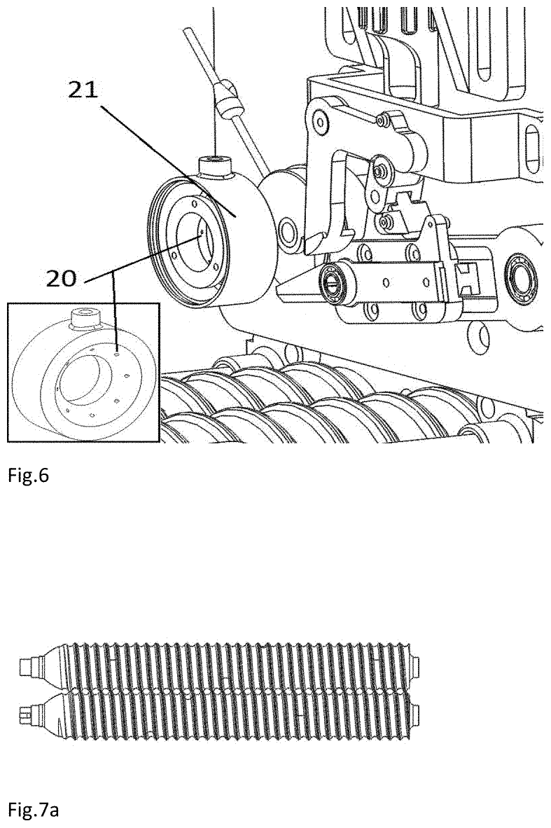

FIG. 7a illustrates the system for forming the extrudates by means of 2 Archimedean screws and FIG. 7b illustrates the axis of rotation induced on the solid forms by slight axial offset of the screws and the gap angle.

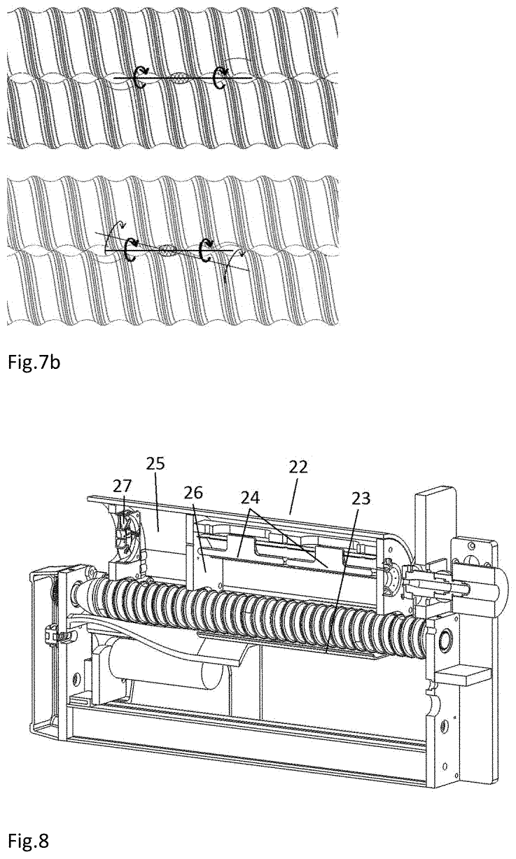

FIG. 8 shows a section in the hot forming system illustrating the heating and cooling zones covering each forming screw.

FIG. 3 shows the chopped part of the molten mass starting from the die (4) by means of the actuating mechanism (6) actuated by a linear motor (7). The die is a cylindrical part connected to the extruder (3), having a circular orifice (8) between 0.5 and 15 mm in diameter depending on the appropriate solid forms to be obtained and preferably of 6 mm for the solid forms comprising an active substance. The die discharge surface is made up of two stepped planes (9 and 10). The first plane (9) is located above the die orifice (8). This plane is in sliding contact with a knife (5), which also allows a clean cut of the rod on actuation of the knife. Underneath the die orifice, a second plane (10), parallel to the first but set back relative to the die orifice, allows the knife to release the chopped rod. This arrangement prevents the chopped rod adhering to the die and thus facilitates its detachment and falling toward the gap of the Archimedean screw. FIG. 3 shows the position of the knife (5) at the end of travel (C) at the moment of ejection of the solid form toward the gap of the Archimedean screw.

The actuating system (6) of the knife (5) has been specially designed so that its return movement to the initial position (A) located above the orifice (8) of the die (4), i.e. ready to chop off the next solid form, does not come into contact with the continuously extruded rod. For this purpose, the knife (5) is mounted on a sliding arm (11—FIG. 4) with a compression spring (12) that pushes the knife (5) to the maximum of its forward position. When the sliding arm (11) swivels upward about its spindle (13) to return the knife to the initial position before chopping (A), a cam (14) that is integral with the knife (5) will come up against a pawl (15) that swivels about a spindle (16).

FIG. 5 illustrates the actuating mechanism and the movement of the chopping device

In fact, during the upward movement of the knife (5), the pawl (15) cannot swivel owing to the stop (17). The pawl (15) therefore forces the knife (5) to move in its slide (11), compressing its spring (12). This backward movement of the knife to position (B) allows it to avoid coming into contact with the extruded rod, as illustrated in FIG. 5e. After a sufficient upward travel, the cam (14) is no longer in contact with the pawl (15) owing to a shoulder in the pawl (18—FIG. 5c and d). The spring (12) of the knife pushes the latter forward in the slide (11) to return it to its position before chopping (A).

During the downward movement of the knife (5) to initiate a new chopping operation, the cam (14) comes up against the upper face (19) of the pawl (15). The latter may then swivel counterclockwise, which is not constrained by the stop (17). The pawl (15) is retracted and thus allows the knife (5) to descend freely for the next chopping operation of the rod, to its position after chopping (C).

The linear motor (7—FIG. 3) may be adjusted for acceleration and for frequency of actuation. These two settings allow adaptation to the cutting force required, which may vary with the rod discharge temperature, and to the rate of extrusion in order to achieve a length of cut and therefore a weight corresponding to the specified objective. The latter is generally of the order of 500 mg with possible variations of some hundreds of mg.

When the rod leaves the die (4) and is readied for chopping by the knife (5), it requires slight surface cooling to limit the risks of adhesion on the knife on the one hand and on the two Archimedean screws on the other hand. The surface cooling of the rod is performed by an annular blowing system (21) positioned opposite the die (4) and concentric with the latter. This system is shown in FIG. 6. It comprises a series of 8 small openings (20) communicating via a common manifold (21). The 8 openings are focused on the outlet of die (8).

The oral form is formed by two corotating Archimedean screws preferably made of stainless steel (FIG. 7a).

The two screws are identical. The length of the screws is between 400 and 1000 mm, preferably 550 mm, the diameter is between 30 and 100 mm and is preferably 50 mm, the pitch may be between 2 and 20 mm and depends on the size of the pellets, but is preferably 14 mm. The depth of the flights varies between 1 and 10 mm, and is preferably 3.6 mm. The shape of the flights makes it possible to produce almost spherical solid forms. The relative angular position of the two screws, and consequently the axial offset of the flights relative to one another, may be adjusted so as to produce axes of rotation on the solid forms additional to the main rotation axis parallel to the axis of the screws. These secondary rotations that are induced generate a movement on the solid forms that is favorable to the generation of spherical shapes (FIG. 7b). They help to remove the mark from chopping of the rod and thus eliminate any surface roughness, which is proscribed for ingestion of the oral form. Typically, the relative angular position of the two screws varies between 0° and 20°.

However, it is difficult to remove this roughness completely, and FIG. 8 illustrates a system (22) for heating to a temperature between 70 and 150° C., obtained for example with infrared heaters (24), which are mounted above the first part of the forming system in order to limit rapid cooling of the solid form coming into contact with the screw. Owing to this heating system, increased malleability of the solid form is ensured in its initial forming phase. A cooling zone (25) obtained by inlet of air at room temperature is provided downstream of this heating zone, in order to achieve a sufficient solid consistency of the solid forms at the end of the travel of the screws and at the moment when they are ejected from the system.

The heating zone (22) is isolated by side walls by means of an upper outer cover and a cover under the screws (23). Inside the heating zone, two IR heaters (24) are placed above the Archimedean screw. The air cooling zone (25) is isolated from the heating zone by a wall (26) that is movable and adjustable. The cooling in the cooling zone is provided by a fan (27) aspirating air via the gap between the screws and discharging it to the surroundings.

### EXAMPLE 1

### Use of the Device for Preparing an Oral Solid Form Comprising Itraconazole

A 150-g premix comprising 25 wt % of itraconazole, 72.5 wt % of pharmaceutical-grade water-soluble polymer soluplus (poly(ethylene glycol grafted with a copolymer of polyvinyl caprolactam and polyvinyl acetate) and with a molecular weight of 110 000 g/mol and 2.5% of AcDisol superdisintegrant (sodium bicarbonate and poloxamer) for easier dissolution of the active substance starting from the solid form, is fed into the extruder using a feed system at a speed of 6 rpm. The mixture of itraconazole with the water-soluble polymer is heated to a temperature of 155° C. in the extruder provided with 2 rotating screws with a length of 550 mm and rotating at a speed of 150 rpm. A temperature gradient is applied to the mixture all the way along the bar of the extruder by the heating system divided into 5 heating zones at the following temperatures: 0-0-140-150-160° C. The temperature of the amorphous mass leaving the extruder is 90′C and is controlled by means of the air-stream cooling system at a pressure of 2 atm.

The extrudate is then delivered in semisolid oral form by means of the chopping device described above at a rate of 0.3 m/s and a frequency of 1 oral solid form per second by means of a linear motor.

The solid form obtained after thermoforming has a mass of 392 g with a relative standard deviation of 3%, which complies with the norms of less than 5% stipulated by the European Pharmacopeia. The average content of active substance is 98 mg with a standard deviation of 1.5%, which also complies with the norms of less than 15% of the pharmacopeia. The spheres obtained no longer have a pronounced mark associated with cutting by the knife.

### EXAMPLE 2

### Use of fhe Device for Preparing an Oral Solid Form Comprising a Food Supplement, Curcumin

A 150-g premix comprising 15, 25 or 35 wt % of curcumin (from Bioextract) and 85, 75 or 65 wt % of Eudraguard EPO (poly(butylnnethacrylate-co-(2-dinnethylanninoethyl)nnethacrylate-co-methyl methacrylate polymer soluble at pH<5 of food grade and with a molecular weight of 47 000 g/mol) for increasing the solubility of curcumin by amorphization starting from the crystalline form of the latter, is fed into the extruder using a feed system at a speed of 4 rpm. Mixing of the curcumin with the Eudragard EPO thermoplastic polymer is carried at a temperature of 155° C. in the extruder provided with 2 corotating screws with a length of 550 mm and rotating at a speed of 100 rpm.

A temperature gradient is applied to the mixture all the way along the extruder barrel by the heating system divided into 5 heating zones at the following temperatures: 0-0-140-150-160° C. The temperature of the amorphous mass leaving the extruder is 90° C. and is controlled by means of the air-stream cooling system at a pressure of 2 atm.

The extrudate is then delivered in semisolid oral form by means of the chopping device described above at a rate of 0.3 m/s and a frequency of 1 oral solid form per second by means of a linear motor provided with a reciprocating piston.

The solid form obtained after thermoforming has a mass of 500 mg with a relative standard deviation of less than 5%. The average curcumin content is 75 mg (if 15% of curcumin), 125 mg (if 25% of curcumin) or 175 mg with a standard deviation of less than 5%. The spheres obtained no longer have a pronounced mark associated with cutting by the knife.

1. A device for preparing continuously solid forms, the device comprising:
an extruder configured to mix and hot melt at least one component with at least one polymer for amorphous solution;
a chopper mounted downstream of the extruder, wherein the chopper is configured to chop a semisolid rod resulting from melting the mixture of the component with the polymer for amorphous solid solution; said chopper being configured with a forming system configured to shape the solid form delivered by the chopper,
wherein the chopper comprises an actuating system of a knife, said actuating system being associated with a motor with an alternating piston actuating a to and fro movement of the knife between two extreme positions, an initial position before chopping and a final position after chopping.

2. The device as claimed in claim 1, wherein the chopper is mounted on a die of the extruder.

3. The device as claimed in claim 1, wherein the chopper is also arranged with a cooling system oriented toward the surface of the semisolid rod, wherein the device is capable of producing solid forms at a controlled temperature.

4. The device as claimed in claim 3, wherein the cooling system comprises a blower arranged downstream of a die of the extruder.

5. The device as claimed in claim 3, wherein the cooling system comprises an annular air jet provided with openings focused on an orifice of the die.

6. The device as claimed in claim 1, wherein the knife of the actuating system is mounted on a sliding arm with a compression spring that is arranged for pushing the knife back along the sliding arm.

7. The device as claimed in claim 6, further comprising a fixed spindle, about which the sliding arm swivels.

8. The device as claimed in claim 1, wherein the actuating system also comprises a pawl provided with a shoulder allowing the knife to avoid the rod in its return movement to the initial position before chopping.

9. The device as claimed in claim 8, wherein the pawl is arranged to swivel about a spindle as far as a stop.

10. The device as claimed in claim 1, wherein the knife further comprises a cam arranged for sliding on a shoulder of a pawl during return of the knife to its position before chopping.

11. The device as claimed in claim 1, wherein the to and fro movement of the knife takes place at a frequency of between 0.1 and 1 m/s.

12. The device as claimed in claim 1, wherein the forming system comprises two corotating Archimedean screws with an angular displacement between 0° and 20°.

13. The device as claimed in claim 1, wherein the forming system is a hot forming system.

14. The device as claimed in claim 1, wherein the mixture comprises a thermoplastic polymer.

15. The device as claimed in claim 1, wherein the mixture comprises a component and a water-soluble polymer whose glass transition temperature (Tg) is below 200° C. and at least 50° C. below the degradation temperature of the polymer and melting of said mixture at a temperature below 250° C.

16. A method of producing an oral solid form comprising an active substance with low water solubility, the method comprising using the device according to claim 1.

17. The method of claim 16, wherein the oral solid form comprises itraconazole.

18. The method of claim 16, wherein the oral solid form comprises a food supplement.

19. The method of claim 18, wherein the food supplement is curcumin.
